(19) United States
(12) Patent Application Publication
(10) Pub. No.: US 2012/0065742 A1
(43) Pub. Date: Mar. 15, 2012

(54) ROTATION STRUCTURE OF KNEE JOINT OF ARTIFICIAL LIMB

(76) Inventor: XIANG-MING WU, New Taipei City (TW)

(21) Appl. No.: 13/228,472

(22) Filed: Sep. 9, 2011

(30) Foreign Application Priority Data
Sep. 15, 2010 (TW) 099217812

(51) Int. Cl. A61F 2/62

(52) U.S. Cl. 623/39

(57) ABSTRACT

A rotation structure of knee joint of artificial limb includes a rotation head forming a rotation-head bore and having a top surface forming a rotation-head recess. The rotation-head recess has an open notch extending outside the rotation head. An arrest block is received in the rotation-head recess and has an end forming a push tab corresponding to the open notch and also forms, in a central portion thereof, an arrest-block opening. A detent is formed on an inner edge of the arrest-block opening. The arrest block is shaped to correspond to the rotation-head recess, but the rotation-head recess has a length greater than that of the arrest block. An arrest shaft forms a retention slot and has a lower end receiving through the arrest-block opening and the rotation-head bore in such a way that the detent of the arrest-block opening is engageable with the retention slot of the arrest shaft.

### TECHNICAL FIELD OF THE INVENTION

The present invention generally relates to a rotation structure of knee joint of artificial limb, which comprises a rotation head forming a rotation-head recess that operates in combination with a detent of an arrest block and a retention slot of an arrest shaft to realize control of a rotatable condition and a latched condition.

### DESCRIPTION OF THE PRIOR ART

A conventional knee joint of artificial limb is operable through a single-axis or multi-axis rotating movement to drive a linkage for bending or stretching movement so as to allow an artificial limb user of above-knee amputation to carry out basic walking activity of bending and stretching. Such a design of knee joint of artificial limb is effective in maintaining safe and stable operation of the knee joint of artificial limb, yet it imposes a limitation to the freedom of a portion of the limb.

The joint movement of a nature lower limb requires coordination among a hip joint, a knee joint, and an ankle joint in order to carry out bending movements of various angles and such a movement comprises bending, stretching, and coaxial rotation of limb. In the conventional design of regular knee joint of artificial limb, a limitation is imposed to the coaxial rotation of limb in order to ensure safe and stable walking. Thus, such a conventional design of knee joint of artificial limb cannot satisfy the needs for rotation of sole and shank at the time when an artificial limb is in a standstill condition. Apparently, further improvements are needed.

### SUMMARY OF THE INVENTION

In view of the above problems, the present invention aims to provide an improvement of the conventional knee joint for artificial limbs, whereby, the knee joint of the artificial limb, when set in a standstill condition, is adjustable of an angular position thereof in coaxial relation to the limb.

The present invention provides a rotation structure of knee joint of artificial limb, which comprises a rotation head forming a rotation-head bore and having a top surface forming a rotation-head recess. The rotation-head recess has an open notch that extends outside of the rotation head. An arrest block is received in the rotation-head recess and has an end forming a push tab corresponding to the open notch of the rotation-head recess and also forms, in a central portion thereof, an arrest-block opening. A detent is formed on an inner edge of the arrest-block opening. The arrest block is shaped to correspond to the rotation-head recess, but the rotation-head recess has a lengthwise dimension that is greater than a lengthwise dimension of the arrest block. A spring and a guide bar are arranged under the arrest block. The push tab of the arrest block is set to correspond to the open notch of the rotation-head recess. An arrest shaft forms a retention slot and has an upper end coupled to a rotary top seat and a lower end receiving through the arrest-block opening and the rotation-head bore in such a way that the detent formed on the inner edge of the arrest-block opening is engageable with the retention slot of the arrest shaft. A needle bearing and a ball bearing are arranged between the arrest shaft and the rotation-head bore. The rotation head comprises a lock washer and a nut arranged at the bottom thereof.

When the rotation structure of knee joint of artificial limb is in a latched condition, the arrest block that is received in the rotation-head recess is acted upon by the spring force of the spring to have the detent formed at the inner edge of the arrest-block opening fit into and engaging the retention slot of the arrest shaft, so that the rotation head 1 is set in a latched condition and is rotatably fixed. To release the latched condition, a user depresses the push tab of the arrest block to deform the spring, and at the same time, the arrest block also removes the detent that is formed on the inner edge of the arrest-block opening out of the retention slot of the arrest shaft, whereby the latched condition of the rotation head is released to allow of angular displacement.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a top plain view of the present invention.

FIG. 4 is a cross-sectional view taken along line A-A of FIG. 3.

FIG. 5 is a cross-sectional view taken along line B-B of FIG. 3.

FIG. 6 illustrates an operation of the present invention.

FIG. 6A is a cross-sectional view of FIG. 6.

FIG. 7 illustrates the operation of the present invention.

FIG. 7A is a cross-sectional view of FIG. 7.

FIG. 8 is a perspective view illustrating another embodiment according to the present invention.

FIG. 9 is an exploded view illustrating said another embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

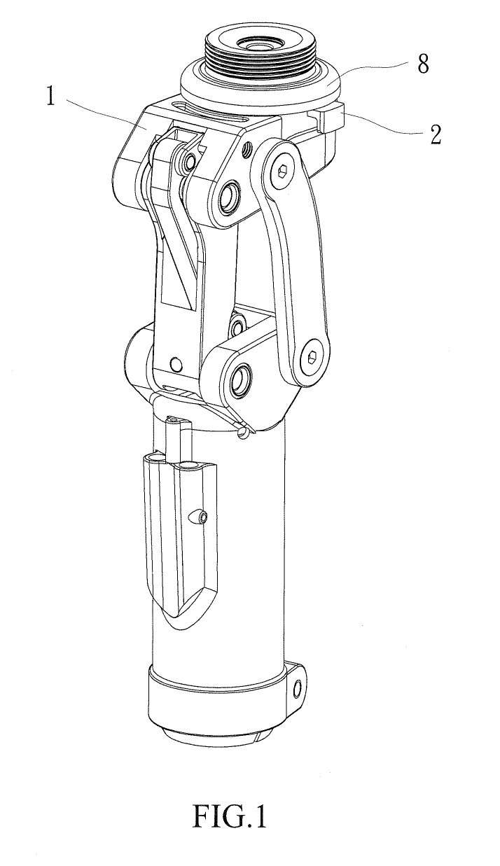

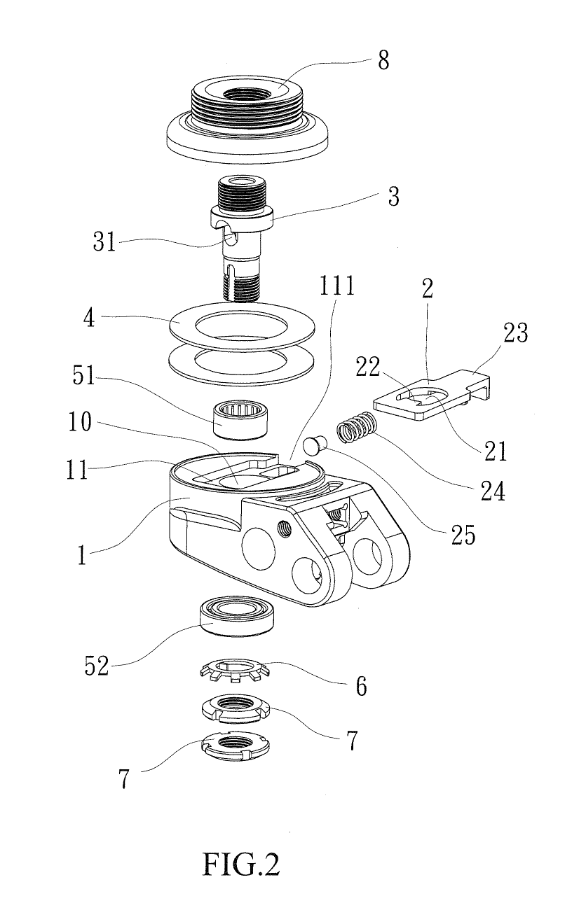

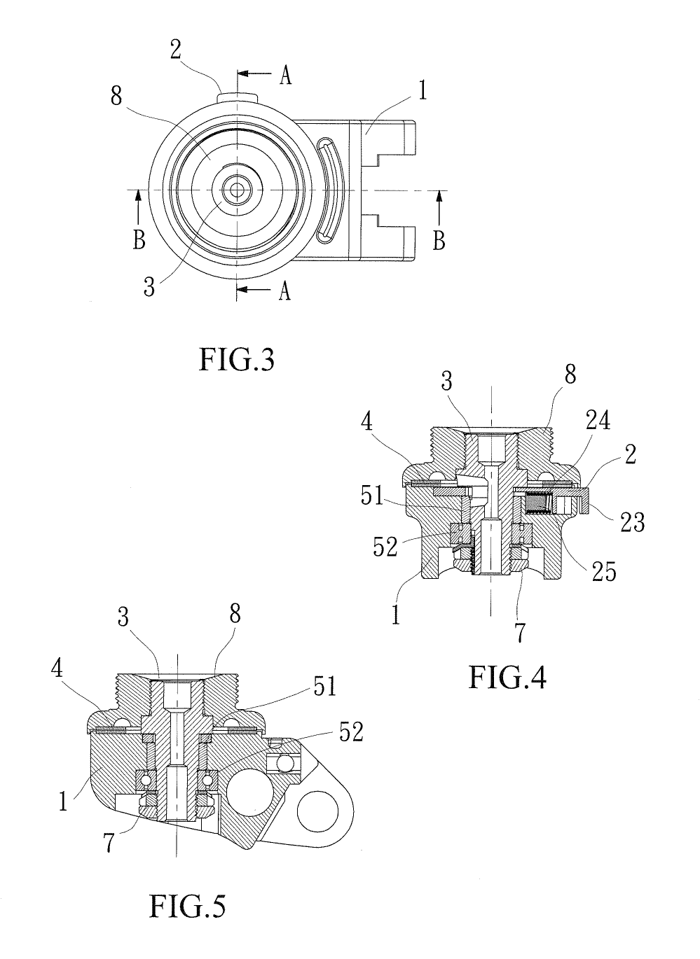

A detailed description of the structure and function of the present invention will be given below with reference to a preferred embodiment thereof in order to facilitate understanding of the present invention. The present invention provides a rotation structure of knee joint of artificial limb, which generally comprises, as shown in FIGS. 1-9, a rotation head 1, an arrest block 2, and an arrest shaft 3.

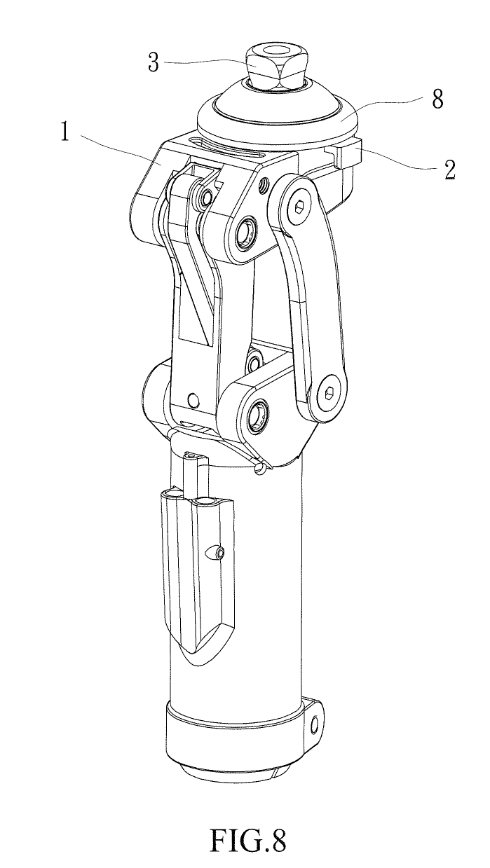

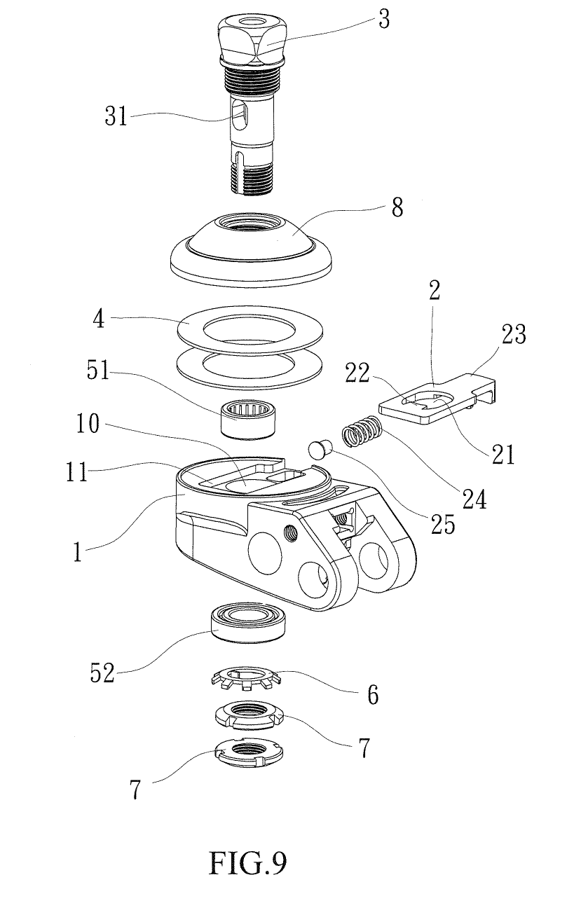

Referring to FIGS. 1-5, the rotation head 1 forms a rotation-head bore 10 and is connected to a link and pneumatic cylinder at the bottom thereof. The rotation head 1 has a top surface in which a rotation-head recess 11 is formed. The rotation-head recess 11 comprises an open notch 111 and the open notch 111 extends to outside of the rotation head 1. The arrest block 2 is received in the rotation-head recess 11 and has an end forming a push tab 23 corresponding to the open notch 111 of the rotation-head recess 11 and also forms, in a central portion thereof, an arrest-block opening 21. A detent 22 is formed on an inner edge of the arrest-block opening 21. The arrest block 2 is shaped to correspond to the rotation-head recess 11, but the rotation-head recess 11 has a lengthwise dimension that is greater than a lengthwise dimension of the arrest block 2. Further, a spring 24 and a guide bar 25 are arranged under the arrest block 2. The arrest shaft 3 forms a retention slot 31 and has a lower end received through the arrest-block opening 21 and the rotation-head bore 10 in such a way that the detent 22 formed on the inner edge of the arrest-block opening 21 is engageable with the retention slot 31 of the arrest shaft 3 and an upper end coupled to a rotary top seat 8. The rotary top seat 8 can be alternatively constructed and presenting the spatial relationship with the arrest shaft 3 as what shown in FIGS. 8 and 9. A needle bearing 51 and a ball bearing 52 are arranged between the arrest shaft 3 and the rotation-head bore 10. The rotation head 1 comprises a lock washer 6 and at least one nut 7 arranged at the bottom thereof. The rotary top seat 8 and the rotation head 1 may receive one or more washers 4 interposed therebetween.

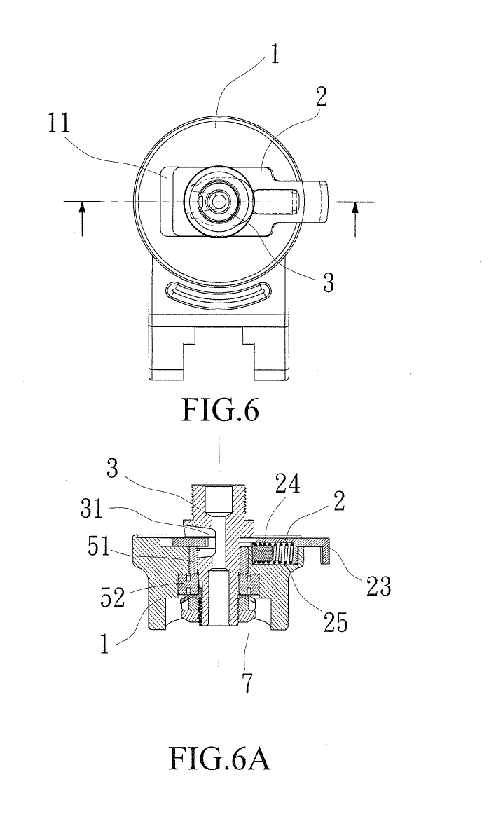

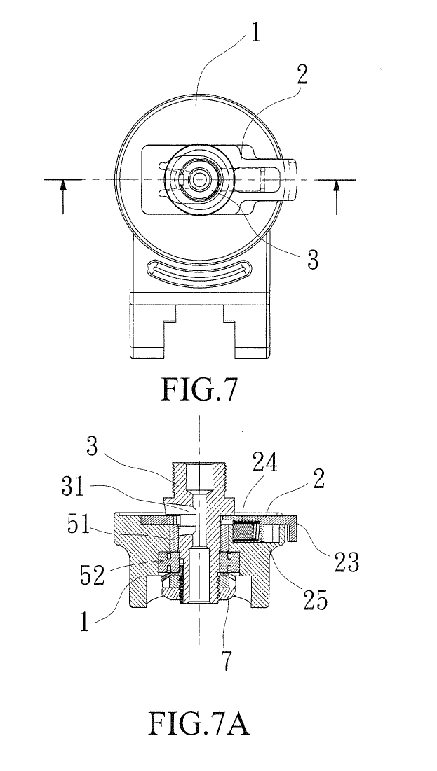

Referring to FIGS. 6, 6A, 7 and 7A, the arrest block 2 and the rotation-head recess 11 have corresponding shapes, but the lengthwise dimension of the rotation-head recess 11 is greater than the lengthwise direction of the arrest block 2, whereby in pushing the push tab 23 under properly arranged spring 24 and guide bar 25, the arrest block 2 is allowed to move within the rotation-head recess 11 and may automatically return. When the rotation structure of knee joint of artificial limb is in a latched condition, the arrest block 2 that is received in the rotation-head recess 11 is acted upon by the spring force of the spring 24 to have the detent 22 formed at the inner edge of the arrest-block opening 21 fit into and engaging the retention slot 31 of the arrest shaft 3. Under this condition, the arrest block 2 arranged in the rotation-head recess 11 sets the rotation head 1 in a latched and rotatably-fixed condition. To release the latched condition, a user depresses the push tab 23 to cause the arrest block 2 to deform the spring 24, and at the same time, the arrest block 2 also removes the detent 22 that is formed on the inner edge of the arrest-block opening 21 out of the retention slot 31 of the arrest shaft 3, whereby the rotation head 1 is released from the latched condition and is allowed to cause a shank and sole of the artificial limb to rotate for realizing adjustment of angle thereof.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A rotation structure of knee joint of artificial limb, comprising: a rotation head, which forms a rotation-head bore and has a top surface forming a rotation-head recess; an arrest block, which is received in the rotation-head recess and has a central portion forming an arrest-block opening, the arrest-block opening having an inner edge forming a detent, the arrest block having a shape corresponding to the rotation-head recess; and an arrest shaft, which forms a retention slot and has an upper end coupled to a rotary top seat and a lower end received through the arrest-block opening and the rotation-head bore, the detent on the inner edge of the arrest-block opening being engageable with the retention slot formed in the arrest shaft.

2. The rotation structure of knee joint of artificial limb according to claim 1, wherein the arrest block is received in the rotation-head recess, the rotation-head recess having a lengthwise dimension greater than a lengthwise dimension of the arrest block.

3. The rotation structure of knee joint of artificial limb according to claim 1, comprises a spring and a guide bar arranged at a lower side.

4. The rotation structure of knee joint of artificial limb according to claim 2, comprising a spring and a guide bar arranged at a lower side.

5. The rotation structure of knee joint of artificial limb according to claim 1, wherein the rotation-head recess comprises an open notch, the open notch extending outside of the rotation head.

6. The rotation structure of knee joint of artificial limb according to claim 1, wherein the arrest block has an end forming a push tab corresponding to the open notch of the rotation-head recess.

7. The rotation structure of knee joint of artificial limb according to claim 5, wherein the arrest block has an end forming a push tab corresponding to the open notch of the rotation-head recess.

8. The rotation structure of knee joint of artificial limb according to claim 1, wherein a needle bearing and a ball bearing are arranged between the arrest shaft and the rotation-head bore and a lock washer and at least one nut are arranged at a bottom of the rotation head.
